(19) United States
(12) Patent Application Publication
(10) Pub. No.: US 2007/0292848 A1
(43) Pub. Date: Dec. 20, 2007

(54) Hormone Receptor Genes and Migraine Susceptibility

(76) Inventors: Lynette Robyn Griffiths, Queensland (AU); Natalie Jane Colson, New South Wales (AU); Rod A. Lea, Wellington (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/571,879

(22) PCT Filed: Sep. 15, 2004
(86) PCT No.: PCT/AU04/01248

§ 371 Date: Jan. 29, 2007

(30) Foreign Application Priority Data
Sep. 15, 2003 (AU) 2003905020

(51) Int. Cl. C12Q 1/68

(52) U.S. Cl. 435/6

(57) ABSTRACT

A method of determining whether an individual has a predisposition to migraine is provided, which method includes the step of isolating a nucleic acid that has a guanine to adenine polymorphism at nucleotide 2014 of a human estrogen receptor and/or a nucleic acid that has a 306 base pair insertion in intron 7 of a human progesterone receptor gene. The presence of either polymorphism is indicative of an increased predisposition to migraine. Furthermore, the presence of both the human estrogen receptor polymorphism and the human progesterone receptor gene polymorphism indicates a three-fold greater predisposition to migraine. Also provided are kits for use with these methods, the kits comprising primers and, optionally, a restriction endonuclease such as Btg1, for molecular detection of a genetic predisposition to migraine.

### FIELD OF THE INVENTION

THIS INVENTION relates to identifying a genetic predisposition to migraine. More particularly, this invention relates to identification of a polymorphism in an estrogen receptor gene and/or in a progesterone receptor gene that is associated with an increased predisposition to migraine and uses thereof for detection of a genetic predisposition to migraine.

### BACKGROUND OF THE INVENTION

Migraine is a common disorder with variable expression (Lance, 1993, Mechanism and management of headache 5th Edition. London: Butterworth Scientific). The exact cause is unknown and there are no recognisable diagnostic pathological changes. Diagnosis is based on symptoms and their groupings. The lack of clear symptom definitions and precise diagnostic criteria, has led to variability in diagnosis. The International Headache Society (Headache Classification Committee of the International Headache Society, 1988, Cephalalgia 8 Supp. 7: 19-28) has, however, recently prepared a new classification for headaches that has made diagnosis clearer and more precisely defined. This system uses the presence of specific attributes to establish diagnosis. The two main types of migraine are termed migraine without aura, previously known as common migraine, and migraine with aura, previously termed classical migraine. Migraine without aura is characterised by recurrent headache, lasting 4-72 hours, with at least two of the following attributes: unilateral location, pulsating quality, moderate to severe intensity and/or aggravation by physical activity. It is also associated with nausea and/or vomiting, or with photophobia and phonophobia. At least 5 attacks of headache fulfilling these criteria are required to separate this type of migraine from episodic tension-type headache. Migraine with aura is characterised by neurological symptoms that usually precede or accompany headache. These symptoms develop over 5-20 minutes, and usually last less than 60 minutes. They most commonly include visual disorders, unilateral numbness or weakness, and aphasia or other speech disorders (Gilman, 1992, New England J Med 326 1610-1616). Headache, nausea, photophobia and/or phonophobia usually follow these symptoms, with headache lasting 4-72 hours. Over 80% of migraine sufferers have headaches without neurological symptoms, while about 20% suffer from migraine with aura. There are a number of other less common types or sub-types of migraine that are accompanied by distinctive neurological symptoms. These include retinal migraine, in which unilateral visual disorders, which may involve temporary blindness, occur with or without headache; familial hemiplegic migraine (FHM), in which headache is accompanied by prolonged hemiparesis; and acephalgic migraine which can involve a variety of neurologic symptoms without headache.

The age of onset of migraine is varied. In females, the onset of disorder is usually at or shortly after puberty, although many children are also diagnosed as suffering from migraine. Much less frequently, onset occurs in middle life and occasionally onset begins during menopause (Walton, 1985, Diseases of the Nervous System 9th Ed. Oxford University Press, UK).

Once onset begins, the manifestation of the disorder may vary within a individual. The pattern and the clinical features of attacks can vary greatly with age in an individual and also between affected family members. It is not uncommon for an individual at different stages in life, to suffer from migraines that, based on clinical features, would be classified as different diagnostic types. Such variations can also be seen within members of the same family (Eadie & Tyrer, 1985, The Biochemistry of Migraine. MTP Press Ltd, Boston USA). A recent study indicated that 45% of migraine with aura families have migraine without aura cases (Mochi et al., 1993, Cephalalgia 13 389-394). In general, migraine attacks usually decrease in frequency and intensity with increasing age.

The pathogenesis and pathophysiology of migraine are poorly understood. Cerebral blood flow changes, specifically a decrease corresponding to the clinically affected area, have been noted as occurring before or at the onset of aura symptoms, in a number of sub-types of migraine with aura. In migraine without aura, however, regional cerebral blood flow remains normal or slightly increased.

Migraine is diagnosed in about 10% of adults but the disorder may be often undiagnosed and hence prevalence is likely to be higher (Linet et al., 1989, JAMA 261 2211-6). Prevalence rates vary depending on migraine definition and population sampled. Kurtze (Kurtze 1982, Neurology 32 1207-1214) determined a conservative prevalence rate of 10% in the US, while Dalsgaard-Nielsen and Ulrich (Dalsgaard-Nielsen & Ulrich, 1972, Headache 12 168-172) found a Danish prevalence rate of 16-23%. More recent and comprehensive studies have indicated prevalence rates of 16% in the European general population (Rasmussen et al., 1991, J Clin Epidemiol 44 1147-57), while in the US, prevalence was determined to be 4% in children, 6% in adult men and 18% in adult women (Stewart et al., 1992, JAMA 267 64-69). A large Dutch survey revealed that the lifetime prevalence of migraine in women was 33% and the 1-year prevalence in women was 25%. In men, this study showed that the lifetime prevalence was 13.3% and the 1-year prevalence was 7.5% indicating that overall the prevalence of migraine may be even higher than previously reported (Launer et al., 1999, Neurol. 53 537-42).

Migraine shows strong familial aggregation. Approximately 50% of migraine sufferers have an affected first degree relative (Goadsby, et al., 1991, Headache 31 365-371), with familial incidence figures varying from 61% (Dalsgaard-Nielsen & Ulrich, 1972, supra) to 90% (Dalsgaard-Nielsen, 1965, Acta Neurologica Scandinavica 41 287-300) and heritability estimates of 40% to 60% (Honkasalo et al., 1995, Headache 35 70-78). The mode of transmission of migraine is controversial but has generally been believed to be autosomal dominant with reduced penetrance (Pratt, 1967, The Genetics of Neurological Disorder. Oxford University Press. London). Other studies (Mochi et al., 1993, supra) support a common genetic background for migraine with and without aura and indicate that there may be a major gene contributing to the disease. A recent review of migraine twin, spouse and family aggregation studies, strongly suggested that both sub-types of migraine are genetically determined with the mode of inheritance most likely multifactorial. However, autosomal dominant inheritance with reduced penetrance, could not be excluded in either sub-type of migraine (Russell & Olesen, 1993, Cephalalgia 13 245-248). Given the growing evidence that there is a genetic basis for migraine sufferers, there is a need to develop molecular diagnostic tests that are capable of determining whether an individual is predisposed to migraine.

### SUMMARY OF THE INVENTION

The present inventors have unexpectedly discovered that genetic polymorphisms in female sex steroid hormone receptor genes may be associated with or linked to a predisposition to migraine.

The present invention is therefore broadly directed to identification of a genetic predisposition to migraine according to the presence of a polymorphism in a female sex steroid hormone receptor gene such as a human estrogen receptor gene or a progesterone receptor gene.

The invention is also broadly directed to identification of a genetic predisposition to migraine according to the presence of a polymorphism in a female sex steroid hormone receptor protein or fragment thereof, such as a human progesterone receptor.

In a first aspect, the invention provides method of determining whether an individual has a predisposition to migraine including the step of isolating a nucleic acid from said individual that comprises a nucleotide sequence of at least a fragment of a female steroid sex hormone receptor gene, wherein the presence of a polymorphism in said nucleotide sequence indicates that said individual has an increased predisposition to migraine compared to an individual without the polymorphism.

In one embodiment, said nucleotide sequence is of at least a fragment of exon 8 of an estrogen receptor gene, wherein if said nucleotide sequence comprises a polymorphism encoding codon 594 of an estrogen receptor, said individual has an increased predisposition to migraine compared to an individual without the polymorphism.

Suitably, the polymorphism is a guanine to adenine change at nucleotide 2014 of the estrogen receptor (ESR1) gene.

In another embodiment, said nucleotide sequence is of at least a fragment of a progesterone receptor gene, wherein if said nucleotide sequence comprises a 306 base pair insertion in intron 7 of said progesterone receptor gene, said individual has an increased predisposition to migraine compared to an individual without the polymorphism.

In a second aspect, the invention provides a method of determining whether an individual has a predisposition to migraine including the step of isolating from said individual

(i) a first nucleic acid that comprises a nucleotide sequence of at least a fragment of a first female steroid sex hormone receptor gene; and

(ii) a second nucleic acid that comprises a nucleotide sequence of at least a fragment of a second female steroid sex hormone receptor gene;

wherein the presence of a polymorphism in said first nucleotide sequence of (i) and in said second nucleotide sequence of (ii) indicates that said individual has an increased predisposition to migraine compared to that of an individual having a polymorphism in (i) or (ii) alone.

Preferably, said first nucleotide sequence in (i) is of at least a fragment of exon 8 of a human estrogen receptor α gene that encodes codon 594 of an estrogen receptor a protein, said individual has an increased predisposition to migraine compared to an individual without the polymorphism.

Even more preferably, the polymorphism in said first nucleotide sequence in (i) is a guanine to adenine change at nucleotide 2014 of the estrogen receptor (ESR1) gene.

Preferably, said second nucleotide sequence in (ii) is of at least a fragment of a progesterone receptor gene, wherein said nucleotide sequence comprises a 306 base pair insertion in intron 7 of said progesterone receptor gene.

In a third aspect, the invention provides a kit for identifying a predisposition to migraine for use in the method of the aforementioned aspects, said kit comprising one or more primers, probes and, optionally, one or more other reagents for identifying said polymorphism(s).

In a particular embodiment, the kit comprises

(a) primers for nucleic acid sequence amplification of at least a fragment of exon 8 of a human ESR1 gene that encodes codon 594 of an estrogen receptor protein; and/or

(b) primers for nucleic acid sequence amplification of at least a fragment of intron 7 of a human progesterone receptor gene.

The kit may further comprise a Btg1 restriction endonuclease.

In a fourth aspect, the invention provides a method of determining whether an individual has a predisposition to migraine including the step of isolating a progesterone receptor protein, or fragment thereof, which indicates that said individual has a human progesterone receptor gene polymorphism that indicates an increased predisposition to migraine compared to an individual without the polymorphism.

Preferably, the progesterone receptor protein is detected according to an altered expression level that indicates said individual has a 306 base pair insertion in the human progesterone receptor gene.

In a fifth aspect, the invention provides a kit comprising one or more reagents for detecting a progesterone receptor protein according to the fourth aspect.

Suitably, according to the aforementioned aspects, said individual is a male or female human.

Throughout this specification, unless the context requires otherwise, the words “comprise”, “comprises” and “comprising” will be understood to imply the inclusion of a stated integer or group of integers but not the exclusion of any other integer or group of integers.

### DETAILED DESCRIPTION OF THE INVENTION

Migraine is a painful and debilitating disorder that affects up to 18% of the population. It imposes a significant economic burden on society due to the costs of medical care, treatment and lost productivity. Genetic and environmental factors play a role in migraine susceptibility although the pathophysiological mechanisms are unclear. The present inventors have reasoned that genetic variation in hormone receptor genes may effect migraine susceptibility and tested this association.

Polymorphisms in the progesterone receptor (PgR) gene, estrogen receptor (ESR1) gene, and androgen receptor (AR) gene were analysed. Allele and genotype frequencies were compared between the groups by generating contingency tables and incorporating chi-squared statistical analyses.

DNA was amplified using PCR techniques. Genotypes were determined for a 306 base pair insertion in the progesterone receptor gene (PROGINS), a trinucleotide repeat variant in the androgen receptor gene, and a restriction fragment length polymorphism in the estrogen receptor gene (exon 8 codon 594). The restriction fragment length polymorphism in the estrogen receptor gene (exon 8 codon 594) and the 306 base pair insertion in intron 7 of the progesterone receptor gene were found to be genetic risk factors associated with migraine.

This invention therefore sets forth and confirms the hypothesis that one or more polymorphisms in the estrogen receptor gene and progesterone receptor gene, are associated with migraine susceptibility.

This is in distinction to the male sex hormone androgen receptor gene which appears to have no association with migraine.

Furthermore, these polymorphisms in the estrogen receptor gene and the progesterone receptor gene interact genetically to increase the predisposition of an individual to migraine.

The generic term “female sex steroid hormone” is used herein in relation to estrogen and/or progesterone.

It will be appreciated that this definition does not imply that female sex steroid hormones are not present or functional in males and/or that the association between migraine predisposition and polymorphisms in female sex steroid hormone receptor genes is limited to females.

Throughout this specification “predisposed and predisposition” in the context of migraine means that an individual has an increased probability of suffering from migraine and includes situations where said individual is not yet exhibiting clinical symptoms of migraine and where said individual is already displaying migraine symptoms. Furthermore, migraine includes “migraine with aura” (MA) and “migraine without aura” (MO) as hereinbefore described.

The term “gene” is used herein as a discrete nucleic acid unit or region that may comprise one or more of introns, exons, open reading frames, splice sites and regulatory sequences such as promoters and polyadenylation sequences.

The term “polymorphism” is used herein to indicate any nucleotide sequence variation in an allelic form of a gene that occurs in a human population. This term encompasses mutation, insertion, deletion and other like terms that indicate specific types of polymorphisms.

In one embodiment, the present invention provides for determination of a predisposition to migraine according to whether an individual has a polymorphism in an estrogen receptor allele that encodes residue 594 of a human estrogen receptor protein. Said polymorphism, if present, is in exon 8 of an estrogen receptor gene. It will therefore be appreciated that by isolating a nucleic acid corresponding to at least a fragment of exon 8 of an estrogen receptor gene that potentially includes the polymorphic codon, a determination can be made as to whether an individual is predisposed to migraine.

Suitably, the polymorphism is a guanine to adenine change at nucleotide 2014 of an ESR1 gene.

This is a “silent” polymorphism in that the encoded amino acid is not altered.

In another embodiment, the present invention provides for determination of a predisposition to migraine according to whether an individual has a polymorphism in a progesterone receptor allele in the form of a 306 base pair insertion in intron 7. It will therefore be appreciated that by isolating a nucleic acid corresponding to at least the portion of intron 7 that potentially includes the insertion, a determination can be made as to whether an individual is predisposed to migraine.

In the context of the present invention by “corresponds to” and “corresponding to” is meant that the isolated nucleic acid comprises a nucleotide sequence of at least a fragment of exon 8 of the estrogen receptor gene that includes codon 594 or, potentially, the relevant insertion within intron 7 of the progesterone receptor gene.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native or recombinant form.

By “protein” is meant an amino acid polymer. The amino acids may be natural or non-natural amino acids, D- or L-amino acids as are well understood in the art.

A “peptide” is a protein having less than fifty (50) amino acids.

A “polypeptide” is a protein having fifty (50) or more amino acids.

The term “nucleic acid” as used herein designates single- or double-stranded mRNA, RNA, cRNA and DNA inclusive of cDNA and genomic DNA and DNA-RNA hybrids.

A “polynucleotide” is a nucleic acid having eighty (80) or more contiguous nucleotides, while an “oligonucleotide” has less than eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

The terms “anneal”, “hybridize” and “hybridization” are used herein in relation to the formation of bimolecular complexes by base-pairing between complementary or partly-complementary nucleic acids in the sense commonly understood in the art. It should also be understood that these terms encompass base-pairing between modified purines and pyrimidines (for example, inosine, methylinosine and methyladenosine) and modified pyrimidines (for example thiouridine and methylcytosine) as well as between A,G,C,T and U purines and pyrimidines. Factors that influence hybridization such as temperature, ionic strength, duration and denaturing agents are well understood in the art, although a useful operational discussion of hybridization is provided in to Chapter 2 of CURRENT PROTOCOLS IN MOLECULAR BIOLOGY (Eds. Ausubel et al. John Wiley & Sons NY, 2000), particularly at sections 2.9 and 2.10.

Diagnostic Methods

The present invention provides methods for determining whether an individual is predisposed to migraine.

Suitably, said individual is a male or female human.

Such methods may be used independently of clinical diagnosis or may be used in conjunction therewith to confirm or assist clinical diagnosis of migraine, inclusive of migraine with aura and migraine without aura.

It will also be appreciated that detection of the ESR1 gene polymorphism and the progesterone receptor gene polymorphism may be performed independently or together.

In a particular aspect, the presence of the ESR1 gene polymorphism and the progesterone receptor gene polymorphism in an individual are indicative of an increased predisposition to migraine compared to that associated with the ESR1 gene polymorphism or the progesterone receptor gene polymorphism alone.

Generally, the methods of the invention are nucleic acid-based methods, given that the female steroid sex hormone receptor polymorphisms described herein have initially been identified and confirmed at the nucleic acid level.

Furthermore, the 594 codon polymorphism is silent with regard to the encoded alanine, hence protein-based analysis of this polymorphism is not contemplated as a preferred form of the invention.

However, it is postulated that the 306 bp Alu repeat insertion in the progesterone receptor gene may affect protein expression, hence protein based methods of detection may be used according to the present invention.

Such methods are well known in the art and include western blotting, ELISA, two dimensional protein profiling, protein arrays, immunoprecipitation, radioimmunoassays and radioligand binding, although without limitation thereto.

With regard to nucleic acid detection, an isolated nucleic acid corresponding to at least a fragment of a female sex steroid hormone receptor gene may be isolated from any appropriate source of nucleic acid, such as lymphocytes or any other nucleated cell type, preferably obtainable by a minimally-invasive method.

The at least a fragment of the isolated nucleic acid may be in the form of genomic DNA, RNA or cDNA reverse-transcribed from isolated RNA.

It will be appreciated that according to the invention, nucleic acid fragments of a female steroid sex hormone gene, or corresponding isolated nucleic acid, suitably comprise less than 100% of the gene or corresponding isolated nucleic acid.

Typically, in certain embodiments fragments may have at least 9, 15, 20, 50 or up to 80 contiguous nucleotides (such as oligonucleotide primers and probes).

In other embodiments, fragments may have 200, 300, 500 or more contiguous nucleotides (such as PCR amplification products).

Suitably, in one form the fragment comprises a guanine corresponding to nucleotide 2014 of an ESR1 gene.

Suitably, in another form the fragment comprises a 306 nucleotide insertion in intron 7 of a PgR gene.

In particular embodiments of the invention, a fragment may be a product of nucleic acid sequence amplification.

Non-limiting examples of such fragments include a 227 base pair fragment produced by PCR amplification of an ESR1 gene and a 173 base pair fragment or a 479 base pair fragment produced by PCR amplification of a PgR gene.

In this regard, it will be appreciated that preferred diagnostic methods employ a nucleic acid sequence amplification technique.

Suitable nucleic acid amplification techniques are well known to the skilled addressee, and include polymerase chain reaction (PCR) and ligase chain reaction (LCR) as for example described in Chapter 15 of Ausubel et al. supra, which is incorporated herein by reference; strand displacement amplification (SDA) as for example described in U.S. Pat. No. 5,422,252 which is incorporated herein by reference; rolling circle replication (RCR) as for example described in Liu et al., 1996, J. Am. Chem. Soc. 118 1587 and International application WO 92/01813, and Lizardi et al., (International Application WO 97/19193) which are incorporated herein by reference; nucleic acid sequence-based amplification (NASBA) as for example described by Sooknanan et aL, 1994, Biotechniques 17 1077, which is incorporated herein by reference; ligase chain reaction (LCR) as for example described in International Application WO89/09385 which is incorporated by reference herein; and Q-β replicase amplification as for example described by Tyagi et al., 1996, Proc. Natl. Acad. Sci. USA 93 5395, which is incorporated herein by reference; and helicase-dependent amplification as for example described in International Publication WO 2004/02025 which is incorporated herein by reference.

As used herein, an “amplification product” is a nucleic acid produced by a nucleic acid sequence amplification technique.

A preferred nucleic acid sequence amplification technique is PCR.

In embodiments relating to the ESR1 gene polymorphism, PCR-based restriction fragment length polymorphism analysis may be used. In this regard, the silent polymorphism in codon 594 of exon 8 of the ESR1 gene is in the form of a guanine to adenine change at nucleotide 2014, which introduces a Btg1 restriction endonuclease site not ordinarily present at a corresponding position in a wild type ESR1 gene.

In embodiments relating to the PROGINS insertion, an amplification product size of 479 base pairs indicates an allele that comprises the 306 bp insertion; an amplification product size of 173 base pairs indicates an allele that contained the 306 bp insertion. Heterozygotic DNA template will produce a 479 bp and a 173 bp amplification product.

Notwithstanding the foregoing, the invention contemplates other nucleic acid detection methods that may be useful for detecting the ESR1 gene polymorphism and/or PROGINS insertion described herein.

For example, a PCR method that may also be useful is Bi-PASA (Bidirectional PCR Amplification of Specific Alleles), as for example described in Liu et al. 1997, Genome Res. 7 389-399.

Another potentially useful PCR method as allele-specification oligonucleotide hybridization, as for example described in Aitken et al., 1999, J Natl Cancer Inst 91 446-452.

It will also be well understood by the skilled person that identification of the or each polymorphism of the invention may be performed using any of a variety of techniques such as fluorescence-based melt curve analysis, SSCP analysis, denaturing gradient gel electrophoresis (DGGE) or direct sequencing of amplification products.

Melt curve analysis can be performed using fluorochrome-labeled allele-specific probes which form base-pair mismatches when annealing to wild-type DNA strands in heterozygotes. Alternatively, fluorescent DNA-intercalating dyes such as SYBR Green 1 can reveal the presence of these base-pair mismatches by virtue of their lower melting temperature (Tₘ) compared to fully complementary sequences. A useful example of allele-specific melt curve analysis can be found, for example, in International Publication No. WO97/46714.

DGGE also exploits Tₘ differences, but uses differential electrophoretic migration through gradient gels as a means of distinguishing subtle nucleotide sequence differences between alleles. Examples of DGGE methods can be found in Fodde & Losekoot, 1994, Hum. Mutat. 3 83-9 and U.S. Pat. Nos. 5,045,450 and 5,190,856.

The or each polymorphism used according to the invention may also be identified by direct sequencing of a PCR amplification product, for example. An example of nucleic acid sequencing technology is provided in Chapter 7 of CURRENT PROTOCOLS IN MOLECULAR BIOLOGY Eds. Ausubel et al. (John Wiley & Sons NY USA 1995-2001).

In yet another embodiment, mass spectroscopy (such as MALDI-TOF) may be used to identify nucleic acid polymorphisms according to mass. In a preferred form, such methods employ mass spectroscopic analysis of primer extension products, such as using the MassARRAY™ technology of Sequenom.

In a further embodiment, a polymorphic female sex hormone receptor-encoding nucleic acid linked to migraine may be identified by a microarray method of the invention.

Microarray technology has become well known in the art and examples of methods applicable to microarray technology are provided in Chapter 22 of CURRENT PROTOCOLS IN MOLECULAR BIOLOGY Eds. Ausubel et al. (John Wiley & Sons NY USA 1995-2001).

With respect to the present invention, a preferred microarray format comprises a substrate such as a glass slide or chip having an immobilized, ordered grid of a plurality of nucleic acid molecules, such as cDNA molecules, although without limitation thereto.

A microarray would typically comprise a nucleic acid having said estrogen receptor gene polymorphism and/or a nucleic acid having said progesterone receptor gene polymorphism together with control estrogen receptor and progesterone receptor nucleic acids.

Such a microarray could also include a plurality of other nucleic acids indicative of other diseases that have an underlying genetic basis and be useful in large scale genetic screening, for example.

It will be appreciated from the foregoing that the invention contemplates a kit for molecular genetic detection of a predisposition to migraine.

In a particular embodiment, the kit comprises

(a) primers for nucleic acid sequence amplification of at least a fragment of exon 8 of a human ESR1 gene that encodes codon 594 of an estrogen receptor protein; and/or

(b) primers for nucleic acid sequence amplification of at least a fragment of intron 7 of a human progesterone receptor gene.

The kit may further comprise a Btg1 restriction endonuclease.

One or more other reagents are contemplated such as probes for hybridization-based methods and detection reagents useful in enzymatic/colorimetric detection of nucleic acids, although without limitation thereto.

So that the present invention may be more readily understood and put into practical effect, the skilled person is referred to the following non-limiting examples.

### EXAMPLES

### Introduction

ESR1 gene is located on chromosome 6q25.1. It is over 140 kilobases in size and has 8 exons (Iwase et al., 1996, Cancer Letters 108 179-184). ESR1 is expressed in various human brain regions including the hypothalamus, limbic system, hippocampus, cortices of the temporal lobe and the brainstem (Osterlund et al, 2000, Journal of Neurochemistry, 75 1390-1398). It is expressed in serotonin neurons of some species (Bethea et al, 2002, Frontiers in Neuroendocrinology 23 41-100). In addition to alternate splicing mechanisms, different promoters are used to regulate ESR1 in distinct neuronal populations (Osterlund et al, 2000, supra). Along with its role in target gene transcription, ligand activated ESR1 has rapid effects on neuronal excitability via second messenger systems, resulting in a range of cellular effects including changes in Ca²⁺ currents and activation of endothelial nitric oxide synthase (Kelly & Levin, 2001, Trends Endocrinological Metabolism, 12 152-156; Luconi et al., 2002, Journal of Steroid Biochemistry & Molecular Biology, 80 369-381; Chen et al., 1999, The Journal of Clinical Investigation, 103 401-406). As changes in neuronal excitability have been implicated in migraine pathogenesis, we hypothesised that genetic variation in ESRimay impact on expression or function, in turn influencing migraine susceptibility.

One particular ESR1 marker under investigation is a silent polymorphism in codon 594 of exon 8 and consists of a guanine to adenine change at nucleotide 2014. It was first described by Roodi et al., 1995, Journal of National Cancer Institute, 87 446-45.

The investigation described herein was undertaken using a population based case-control approach. Due to past problems with non-replication of positive associations, we have also performed an additional study on an independent population based cohort using the same marker.

The human progesterone receptor gene is located on chromosome 11q22. It exists as 2 functionally distinct isoforms, PRA and PRB. PRB functions as a transcriptional activator of progesterone-responsive genes, while PRA is transcriptionally inactive and functions as a strong ligand-dependent repressor of steroid hormone receptor transcriptional activity (Giangrande et al., 1997, Journal Biological Chemistry, 272 32889-32900). Progesterone receptor (PgR) expression is upregulated by estrogen and down-regulated by progesterone in most target tissues (Bouchard, 1999, Journal of Reproductive Medicine 44 Suppl2 153-157.). The PgR is found in various regions of the human brain including serotonin neurons (Lombardi et aL, 2001, Molecular and Cellular Endocrinology 178 51-55; Bethea et al., 2002. Frontiers in Neuroendocrinology, 23:41-100). Similar to ESR1, PgR can undergo ligand-independent activation and is involved in various intracellular signalling pathways (Cenni & Picard, 1999, Trends Endocrinol Metab 10 41-46).

The PROGINS polymorphic insertion is a 306 base pair insertion that occurs within intron G of the progesterone receptor gene in some individuals. Although it does not occur within a coding region of the PgR gene, it may have a deleterious effect on progesterone receptor expression, through recombination or mis-splicing (Rowe et al., 1995, Cancer Research, 55 2743-2745; Donaldson, et al., 2002, Mutation Research, 501137-141; Kieback et al., 1995, Journal of the Society for Gynecological Investigation, 2 137 Wang-Gohrke et al., 2000, Cancer Research, 60 2348-2350). The PROGINS Alu insertion has been investigated for a possible role in breast cancer. In present study, it has been examined for a possible association with migraine due to its potential role in migraine pathogenesis.

The human AR is located on chromosome Xq11-12 and in humans is expressed in various organs including the brain in both males and females. It includes three major functional domains, the N-terminal domain, which is involved in transcriptional activation of target genes, coded for by exon 1, a cysteine rich DNA binding domain encoded by exons 2&3, and a hormone binding domain, encoded by exons 4-8 (Keller et al., 1996, Trends Endocrinological Metabolism, 12 152-156).

A polyglutamine tract encoded by CAG repeats occurs in Exon 1 of the Androgen Receptor Gene. Expansion of this repeat is considered to have an inhibitory effect on transactivation function due to interaction of this region with various co-activators. Short fragments are associated with enhanced receptor function (Westberg et al., 2001, The Journal of Clinical Endocrinology & Metabolism, 86 2562-2568), while longer CAG repeats decrease AR activity. This reduced activity has been demonstrated to reduce negative feedback to the hypothalamus, resulting in increased serum androgen levels (Krithivas et al., 1999, J. Endocrinol. 162 137-142) Furthermore, abnormal expansions of poly-glutamine tracts in the central nervous system cause neurodegenerative diseases such as Huntingtons disease and spinocerebellar ataxia type 1 (Chamberlain et al, 1994, Nucleic Acids Research, 22 3181-3186). It has been suggested that the effect of polyglutamine repeat length may be gene specific. The activity of the AR may be unaffected on genes that determine sexual differentiation, but compromised on genes necessary for normal neuronal function (Chamberlain et al., 1994, supra). Alleles of different sizes within the considered normal range of the AR CAG repeat have been associated with androgen dependent prostate-cancer (Young et al., 1998, Reviews of Reproduction 3 141-144.), and arterial vasoreactivity in males (Zitmann et al., 2001, Journal Endocrinological Metabolism, 86, 4867-4873). In this study the AR CAG repeat polymorphism will be examined for a potential association with migraine.

### Materials and Methods

Study Population

Research was approved by the Griffith University Ethics Committee for experimentation on human subjects. All 1,150 participants of the study gave informed consent prior to participation. All participants were interviewed, and completed a detailed questionnaire providing information including personal and family medical history, migraine symptoms, age of onset, frequency, severity, and treatment as previously described (Lea et al., 2000, Neurogenetics 3 35-40; Johnson et al., 2003, Am J Med Genet 117B 86-89). This questionnaire revealed that 78% of individuals in the migraine group had a known family history of migraine. Migraineurs were diagnosed by a clinical neurologist as having either MA or MO based strictly on the widely accepted criteria specified by the IHS (Headache Classification Committee of the International Headache Society (1988) Classification and diagnostic criteria for headache disorders, cranial neuralgias and facial pain. Cephalgia 8 Suppl 7 20). The first study population was comprised of 275 migraineurs and 275 unrelated control individuals. The controls were matched for sex, age (±5 years), and ethnicity (Caucasian) to avoid the potential bias of population stratification, and were recruited in parallel at a similar time and geographical location (East Coast of Australia) as the case group. Clinical characteristics of the case group appear in Table 1.

A follow-up second study population consisted of 300 migraineurs similarly diagnosed and matched with 300 controls.

All participants provided a blood sample from which DNA was extracted by a modification of the salting out method used by Miller et al., 1988, Nucleic Acids Res 16 1215.

Genotyping

Genotyping for the ESR1 G594A marker was undertaken by polymerase chain reaction (PCR) and restriction enzyme digestion. Oligonucleotide primers used were those previously described by Curran et al., 2001, Internat. J. Cancer (Pred Oncol), 95 271-275 to produce a 227 bp amplification product:

5′-GAG ACG GAC CAA AGC CAC-3′ (sense; SEQ ID NO:1); and

5′-GCC ATT GGT GTT GGA TGC ATG C-3′ (antisense; SEQ ID NO:2).

The 20 μl PCR reaction mix contained 50 ng genomic DNA, 0.25 μM of each primer, 1×PCR buffer, 3.75 mM MgCl₂, 0.2 mM dNTPs and DNA polymerase. Thermocycler conditions were 94° C. for 2 minutes 30 seconds, 5 cycles of 94° C. for 45 seconds, 69° C. for 1 minute, and 72° C. for 2 minutes, followed by 30 cycles of 94° C. for 30 seconds, 67° C. for 30 seconds and 72° C. for 45 seconds, with a final step of 72° C. for 5 minutes. Following amplification, 10 μl of product was digested with BtgI overnight at 37° C. After digestion, the product was loaded into a 5% Agarose gel stained with ethidium bromide and electrophoresed at 90V for 60 minutes. An undigested sample indicated presence of the 594A allele.

Genotyping for the PR progins insert marker was undertaken by polymerase chain reaction (PCR). Oligonucleotide primers used were those previously described by Lancaster et al., 1998, Br. J. Cancer 78 277. The 20 μl PCR reaction mix contained 30 ng genomic DNA, 0.25 μM of each primer, 1×PCR buffer, 1.5 mM MgCl₂, 0.2 mM dNTPs and DNA polymerase. Thermocycler conditions were 94° C. for 4 minutes, followed by 30 cycles of 94° C. for 30 seconds, 51° C. for 30 seconds and 72° C. for 45 seconds, with a final step of 72° C. for 2 minutes. Following amplification, 10 μl of amplification product was loaded into a 2% Agarose gel using a 100 bp ladder for comparison. The gel was stained with ethidium bromide and electrophoresed at 90V for 60 minutes. An amplification product size of 479 base pairs indicated an allele that contained the 306 bp insertion; an amplification product size of 173 base pairs indicated an allele that contained the 306 bp insertion.

Genotyping for the AR marker was undertaken by polymerase chain reaction (PCR) and capilliary electrophoresis using the ABI 310 Genescan™. Oligonucleotide primers used were those previously described by Sleddens et al 1992, Nucl. Acids. Res. 20 1427. The 15 μl PCR reaction mix contained 50 ng genomic DNA, 0.3 μM of each primer, Optimisation buffer H and DNA polymerase. Thermocycler conditions were 94° C. for 4 minutes, followed by 30 cycles of 94° C. for 60 seconds, 59° C. for 60 seconds and 72° C. for 30 seconds, with a final step of 72° C. for 2 minutes. Following amplification, Genotyping was carried out using the ABI 310 Genescan™ Genotyper computer software which converts the genescan sized peaks into genotype calls using macros.

Statistical Analysis

ESR1: Genotype data and allele frequencies were compared between the two populations using standard chi-squared analysis. Only when results were available from both matched pairs, i.e., migraine-affected and age- and sex-matched controls, were they included in the genotypic analyses. Odds ratios (OR) and 95% confidence intervals (CI) were calculated. Due to multiple testing, the Bonferroni correction for five tests was applied, which set the level of significance at 0.01 (i.e., 0.05/5) (Mantel & Haenszel, 1959, J Natl Cancer Inst 22 719-748). All genotype frequencies were tested for Hardy-Weinberg equilibrium.

PROGINS and AR: Genotype data and allele frequencies were compared between the migraine and unaffected groups using standard chi-square analysis, or CLUMP analysis using the Monte Carlo approach in the case of the AR multiallelic marker. Monte Carlo analysis may be used to analyse markers that result in sparse contingency tables. As recommended by Sham and Curtis, 1995, Annals. Hum. Genet. 59 97-105, we have presented the T1 statistic, which calculates a chi-squared statistic of the raw contingency table, and the T4 statistic, the maximised chi-squared statistic of all possible 2×2 tables (Sham & Curtis, 1995, supra). The Clump program was run over 5000 simulations to estimate P values.

### Results

Estrogen Receptor 1 Gene

Statistical analysis revealed a significant difference between genotyped migraineurs and the matched control group with regard to allele frequencies (P=0.003) and genotype frequencies (P=0.008). Results of comparisons between male case and control groups (allele frequency P=0.034, genotype frequency P=0.046) and female case and control groups (allele frequency P=0.032, genotype frequency P=0.064) indicated that no significant gender effect was evident. Furthermore, the association was seen in both subgroups, MA (allele frequency P=0.013, genotype frequency P=0.025) and MO (allele frequency P=0.019, genotype frequency P=0.007). Consequently, the significant association seen in the case-control analysis occurred similarly in both males and females, and in the MA and MO subgroups. Results are displayed in Table 2.

Results indicated that individuals who carried the 594A allele were 1.8 times more likely to suffer from migraine [OR=1.8, 95% CI=1.2-2.6, p=0.003] than those who did not carry this allele (Table 3).

The follow-up independent study also revealed a significant difference between genotyped migraineurs and the matched control group with regard to allele frequencies (P=8×10⁻⁶) and genotype frequencies (P=4×10⁻⁵). This significant association occurred in females (allele frequency P=3×10⁻⁶, genotype frequency P=2×10⁻⁵) and in the MA subgroup (allele frequency P=1×10⁻⁶, genotype frequency P=7×10⁻⁶). Although the association did not occur in males (allele frequency P=0.717, genotype frequency P=0.127) and the MO subgroup (allele frequency P=0.529, genotype frequency P=0.818), this may be due to small numbers in these subgroups (males n=36, MO n=39). Alternatively, estrogen and its receptor may play a lesser role in male migraineurs. Results are displayed in Table 4. Allele frequencies in both study populations did not deviate from Hardy-Weinberg equilibrium (P=0.14, P=0.88), and each independent sample cohort showed similar frequencies in the case and control groups. Internal controls using random repeat samples and negative controls were used to confirm genotypes and to exclude the potential for genotyping errors, which in our hands have been estimated to be <5%. Only when results for both of a matched pair were obtained, were they included in the analysis. Results of OR calculations based upon the Mantel Haenszel method of combining the datasets (Mantel & Haenszel, 1959, supra), comparing the G/G genotypes with the G/A and A/A genotype frequencies together, indicated that individuals who carried the 594A allele were twice as likely to suffer from migraine (OR=1.96, 95% CI=1.43-2.68) than those who did not carry this allele. Similarly, OR were calculated on the subgroups comparing G/G genotypes with the G/A and A/A genotype frequencies together. Results were as follows: MA subgroup OR=1.97, (95% CI=1.41-2.77); MO subgroup OR=1.80 (95% CI=1.10-2.94); males OR=1.95 (95% CI=0.95-3.98); females OR=1.96 (95% CI=1.39-2.78).

Progesterone Receptor and Androgen Receptor

To analyse whether variation in two migraine candidate gene loci were associated with typical migraine, we tested the CAG repeat in Exon 1 of the AR gene, and the PROGINS insert in the PgR gene by independent cross-sectional association analysis. For all genotype analyses internal controls were carried out using random repeat samples and negative controls.

Progesterone Receptor Gene PROGINS Insert

Results of analysis of the PgR PROGINS variant in study population 1 consisting of 275 migraineurs and 275 unrelated control individuals showed that the PROGINS allele was over-represented in the migraine group compared to healthy controls (genotype frequencies X²=6.50 P=0.04, allele frequencies X²=5.65, P=0.02). Results of the subgroup analysis showed a significant difference in the MO (genotype frequencies X²=13.08 P=0.001, allele frequencies X²=7.06, P=0.008) and the female subgroups (genotype frequencies X²=10.64 P=0.005, allele frequencies X²=8.1, P=0.004), but not the MA (genotype frequencies X²=2.25 P=0.33, allele frequencies X²=2.47, P=0.12) and male subgroups (genotype frequencies X²=0.41 P=0.82, allele frequencies X²=0.27, P=0.60). Frequency distribution appears in Table 5.

This marker was investigated in the independent follow-up population (population 2) of 300 migraineurs and 300 controls. Results showed a significant difference in genotype (X²=7.92, P=0.019) and allele frequencies (X²=8.78, P=0.003) in the total group analysis, and in the MA subgroup (genotype frequencies X²=7.28 P=0.026, allele frequencies X²=7.91, P=0.005). Similar results were seen in both male (genotype frequencies X²=5.27 P=0.07, allele frequencies X²=5.87, P=0.02) and female subgroups (genotype frequencies X²=4.81 P=0.09, allele frequencies X²=4.31, P=0.04), although they did not reach statistical significance. Analysis of the MO subgroup did not reach statistical significance (genotype frequencies X²=3.53 P=0.17, allele frequencies X²=3.11, P=0.08). Frequency distribution appears in Table 6. Allele frequencies in both study populations did not deviate from Hardy Weinberg Equilibrium at P=0.22 and P=0.13 respectively. Published allele frequencies vary somewhat but a recent analysis of this variant in 21 diverse human populations reported an average allele frequency of 11% and a heterozygosity of 0.188 (Donaldson et al., 2003, supra).

In order to analyse whether the PROGINS PgR variant exerts a dominant or recessive effect on migraine susceptibility, we investigated the effect of the genotype risk groups (12/22; 22 only) and found that the 12/22 genotype was significantly over-represented in the total migraine subgroup of both populations (24%) compared to the total control subgroup (14%) (X²=13.94, P=2×10⁻⁴). Odds ratios were calculated using the Mantel Haenszel method of combining the datasets (Mantel & Haenszel, 1959, supra), comparing those who carried the PROGINS allele and those who did not. Results indicated that those who carried the PROGINS allele were 1.8 times more likely to suffer from migraine than those who did not carry this allele (OR=1.77, 95% CI=1.23-2.55).

Androgen Receptor CAG Repeat

As the AR gene occurs on the X chromosome, only one copy exists in males. Therefore all analyses were performed on allele frequencies. Results of analysis of the AR variant showed a borderline difference between affected and control groups producing a T1 X² value of 26.46, and a P value of 0.048. The T4 X² value of 13.02, P=0.13, which was achieved by clumping together alleles 1,2, 3, 5, 6, 9, 10, 11, 13, 15, 16, 17 was not significant. Comparisons of MA v control (T1 X₂=18.74, P=0.28: T4 X²=10.21, P=0.21), male case v control (T1 X²=13.31, P=0.42; T4=8.16, P=0.52), and female case v control (T1 X²=19.48, P=0.25; T4 X²=8.49, P=0.40) were not significant. A significant result was seen in the MO v control comparison (T1 X²=33.26, P=0.01; T4=X²=16.22, P=0.03).

The data were dichotomised based on the mode (17 CAG repeats) and a 2×2 contingency table was generated. Results of the chi-square analysis showed no significant difference in frequencies between cases and controls (P=0.36), MA v control (P=0.83), MO v control (P=0.58), and both male vs control (P=0.35) and female case vs control (P=0.22).

Estrogen Receptor Gene and Progesterone Receptor Gene Interaction

Estrogen, progesterone and their receptors play a complex, interdependent role in the CNS. Because we have found a positive association of the PgR PROGINS insert in this study, as well as an association of the ESR1 G594A polymorphism with migraine in the same study population, we have undertaken interaction analysis to determine if possession of both risk genotypes confers an increased risk of migraine.

Results showed that 30% of all migraineurs carried at least one copy of the risk allele from both ESR1 and PgR genes compared to only 12% of controls. To determine the magnitude of the increased risk of migraine conferred specifically by the risk alleles from both ESR1 and PgR genes, odds ratios were calculated after dichotomising the genotype frequency data into risk (possessing at least 1 copy of the risk allele from each gene) and no risk (possessing zero copies of the risk alleles) groups (Table 7). Comparing the total migraine group against controls (populations 1 and 2 together) under this grouping scheme produced an OR of 3.2 with a 95% CI of 1.9-5.3. Therefore, it appears that the PROGINS allele of PgR gene acts synergistically with the 594A allele of ESR1 to increase the risk of migraine. That is, these alleles act in combination to increase the risk of migraine by a factor of 3, which is greater than the independent effects of these genetic variants on disease susceptibility.

### CONCLUSIONS

We have conducted a case-control association study to investigate the estrogen receptor 1 gene as a candidate in migraine susceptibility. The study tested two large carefully matched case-control populations for an ESR1 exon 8 single nucleotide polymorphism. Results of this study have indicated a positive association of an ESR1 exon 8 polymorphism with migraine in two independent cohorts. This positive association was seen equally in all subgroups in the initial study group and in the female and MA subgroups in the follow-up group. Lack of association in males may have been related to the limited number of males in the second population. However, it is also possible that hormonal factors may play a different role in the two genders in migraine, and estrogen and its receptor may play a lesser role in male migraineurs.

Previous population genetics studies performed in our laboratory have shown that markers in exon 1 and exon 4 were not in linkage disequilibrium with the exon 8 variant (Curran et al., 2001, supra). This information suggests that the migraine susceptibility haplotype is specific to the 3 region of the ESR1 gene. To our knowledge this is the first study to report an association of a hormone-related gene variant with migraine and these data suggest that the estrogen receptor gene may be an integral player in mechanisms that are relevant to migraine pathogenesis.

Results of the AR CAG repeat total group analysis showed an interesting borderline result, although this was primarily due to the MO subgroup. However after applying the Bonferroni correction for multiple testing, which set the level of significance at 0.01 (i.e. 0.05/5), overall results did not reach statistical significance. Furthermore there was no significant difference in allele frequencies when data were dichotomised and analysed using the chi-square statistic.

We also demonstrated significant association of the PgR PROGINS insert with migraine susceptibility. Statistical significance was reached in the first study population of 275 migraineurs compared to healthy controls, and also in the independent follow-up group of 300 migraineurs compared to controls. Furthermore, analysis of genotype risk groups showed that the PROGINS insert allele was significantly over-represented in migraineurs, and those who carried this allele were 1.8 times more likely to suffer migraine. We note that sub-group analysis showed dissimilar results in the MA/MO analyses in the two independent populations, however small numbers in the MO sub-group would have reduced statistical power, and may have contributed to this anomaly. Under the hypothesis of similar genetic etiology in the migraine subgroups, it would be expected that the PROGINS insert would confer a risk in both subgroups.

We also report herein a significant association of the PROGINS PgR insert in the same population. As both genes play a complex, interrelated role in the CNS, and have been independently implicated in migraine susceptibility, we also analysed the impact on migraine risk of carrying susceptibility alleles for both genes. Results showed that individuals who carried a copy of both PgR and ESR1 risk alleles were 3.2 times more likely to suffer from migraine, an effect that is greater than the independent effects of these genetic variants on disease susceptibility. These results present evidence for an interactive role of both variants in migraine susceptibility.

Thus the PROGINS allele interacts synergistically with the ESR1 594A allele to increase the risk of migraine by a factor of 3.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to in this specification are incorporated herein by reference in their entirety.

TABLE 1
Clinical characteristics of the initial case group

| Characteristic | Proportions |
|---|---|
| Gender | 75 Male (27%) 200 Female (73%) |
| Family History | 215 Yes (78.5%) 46 No (16.8%) 14 Unsure (4.7%) |
| Duration | Average 12-24 hours |
| Frequency | Average 1-2 per month |
| Age of Onset | Average 19.8 years |

TABLE 2
Distribution of ESR1 exon 8 codon 594 ACG/ACA polymorphism
frequencies in migraineurs and controls of original sample
(MO migraine without aura, MA migraine with aura)

| Genotypes | | | | n | Alleles | |
|---|---|---|---|---|---|---|
| Group | GG | GA | AA | (alleles) | G | A |
| Migraine | 81 (36%) | 120 (54%) | 23 (10%) | 448 | 282 (63%) | 166 (37%) |
| Male | 18 (32%) | 33 (58%) | 6 (10%) | 114 | 69 (61%) | 45 (39%) |
| MA | 11 (31%) | 19 (53%) | 6 (16%) | 72 | 41 (57%) | 31 (43%) |
| MO | 7 (33%) | 14 (67%) | 0 | 42 | 28 (68%) | 14 (32%) |
| Female | 63 (38%) | 87 (52%) | 17 (10%) | 334 | 213 (64%) | 121 (36%) |
| MA | 44 (43%) | 47 (46%) | 12 (11%) | 206 | 135 (66%) | 71 (34%) |
| MO | 19 (30%) | 40 (62%) | 5 (8%) | 128 | 78 (61%) | 50 (39%) |
| Control | 112 (50%) | 99 (44%) | 13 (6%) | 448 | 323 (72%) | 125 (28%) |
| Male | 28 (49%) | 28 (49%) | 1 (2%) | 114 | 84 (74%) | 30 (26%) |
| Female | 84 (50%) | 71 (43%) | 12 (7%) | 334 | 239 (72%) | 95 (28%) |
| Total case vs. | X² = 9.77 | P = 0.008 | | X² = 8.56 | P = 0.003 | df = 1 |
| control | | | | | | |

TABLE 3
Chi-squared (χ²) analysis of migraine groups for ESR Exon 8
Codon 594 ACG/ACA Polymorphism

| | Frequency comparison | | | |
|---|---|---|---|---|
| Group | Genotypes | | Alleles | |
| Total Case V Control | χ² = 9.77 | p = 0.008 | χ² = 8.56 | p = 0.003 |
| Male V Female | χ² = 0.72 | p = 0.699 | χ² = 0.38 | p = 0.535 |
| Migraineurs | | | | |
| Male Case V Control | χ² = 6.16 | p = 0.046 | χ² = 4.47 | p = 0.034 |
| Female Case V Control | χ² = 5.48 | p = 0.064 | χ² = 4.63 | p = 0.032 |

TABLE 4
Distribution of ESR1 exon 8 codon 594 ACG/ACA polymorphism
frequencies in independent sample

| Genotypes | | | | n | Alleles | |
|---|---|---|---|---|---|---|
| Group | GG | GA | AA | (alleles) | G | A |
| Migraine | 103 (40%) | 125 (48%) | 32 (12%) | 520 | 331 (64%) | 189 (36%) |
| Male | 15 (42%) | 19 (53%) | 2 (5%) | 72 | 49 (68%) | 23 (32%) |
| MA | 11 (37%) | 17 (56%) | 2 (7%) | 60 | 39 (65%) | 21 (35%) |
| MO | 4 (67%) | 2 (33%) | 0 | 12 | 10 (83%) | 2 (17%) |
| Female | 88 (39%) | 106 (47%) | 30 (14%) | 448 | 282 (63%) | 166 (37%) |
| MA | 71 (37%) | 93 (49%) | 27 (14%) | 382 | 235 (62%) | 147 (38%) |
| MO | 17 (52%) | 13 (39%) | 3 (9%) | 66 | 47 (71%) | 19 (29%) |
| Control | 152 (58%) | 93 (36%) | 15 (6%) | 520 | 397 (76%) | 123 (24%) |
| Male | 20 (55%) | 11 (31%) | 5 (14%) | 72 | 51 (71%) | 21 (29%) |
| Female | 132 (59%) | 82 (37%) | 10 (4%) | 448 | 346 (77%) | 102 (23%) |
| Total case vs. | X² = 20.26 | P = 4 × 10⁻⁵ | | X² = 19.95 | P = 8 × 10⁻⁶ | df = 1 |
| control | | | | | | |

TABLE 5
Distribution of PgR PROGINS Polymorphism frequencies in
migraineurs and controls of Association 1

| | Genotypes | | | N | Alleles | |
|---|---|---|---|---|---|---|
| Group | 11 | 12 | 22 | (alleles) | 1 | 2 |
| Migraine | 173 (75%) | 55 (23%) | 4 (2%) | 464 | 401 (86%) | 63 (14%) |
| Male | 43 (64%) | 22 (33%) | 2 (3%) | 134 | 108 (81%) | 26 (19%) |
| Female | 130 (79%) | 33 (20%) | 2 (1%) | 130 | 302 (92%) | 38 (8%) |
| MA | 113 (80%) | 27 (19%) | 4 (3%) | 288 | 253 (88%) | 35 (12%) |
| MO | 60 (68%) | 28 (32%) | 0 (0%) | 176 | 148 (84%) | 28 (16%) |
| Control | 182 (84%) | 31 (15%) | 3 (1%) | 432 | 395 (91%) | 37 (9%) |
| Male | 44 (68%) | 20 (31%) | 1 (1%) | 130 | 108 (83%) | 22 (17%) |
| Female | 138 (91%) | 11 (7%) | 2 (2%) | 302 | 287 (81%) | 15 (5%) |

| | | |
|---|---|---|
| Total Case v Control | χ² = 6.5 p = 0.039 | χ² = 5.65 p = 0.017 |

11 = No Progins insert,
12 = heterozygote,
22 = homozygote for PROGINS insert

TABLE 6
Distribution of PgR PROGINS Polymorphism frequencies in
migraineurs and controls of Association 2

| | Genotypes | | | N | Alleles | |
|---|---|---|---|---|---|---|
| Group | 11 | 12 | 22 | (alleles) | 1 | 2 |
| Migraine | 215 (78%) | 54 (19%) | 8 (3%) | 554 | 484 (87%) | 70 (13%) |
| Male | 27 (69%) | 8 (20%) | 4 (11%) | 78 | 62 (79%) | 16 (21%) |
| Female | 188 (79%) | 46 (19%) | 4 (2%) | 476 | 422 (89%) | 54 (11%) |
| MO | 176 (77%) | 45 (20%) | 6 (3%) | 454 | 397 (87%) | 57 (13%) |
| MA | 39 (78%) | 9 (18%) | 2 (4%) | 100 | 87 (87%) | 13 (13%) |
| Control | 228 (87%) | 32 (15%) | 3 (1%) | 526 | 488 (93%) | 38 (7%) |
| Male | 35 (85%) | 6 (15%) | 0 (0%) | 82 | 76 (93%) | 6 (7%) |
| Female | 193 (87%) | 26 (12%) | 3 (1%) | 444 | 412 (93%) | 32 (7%) |

| | | |
|---|---|---|
| Total Case v Control | x² = 7.92 p = 0.019 | x² = 8.78 p = 0.003 |

11 = No Progins insert,
12 = heterozygote,
22 = homozygote for PROGINS insert

TABLE 7
Distribution of individuals who carried none/both risk alleles

| Migraine | | At least one risk allele |
|---|---|---|
| Diagnosis | No risk Alleles | from each gene |
| Migraine | 132 (70%) | 57 (30%) |
| Control | 191 (88%) | 26 (12%) |
| | X² = 20.53 p = 3 × 10⁻⁵ | |

1. A method of determining whether an individual has a predisposition to migraine comprising
obtaining a biological sample from said individual said sample comprising at least one nucleic acid from said individual that comprises a nucleotide sequence of at least a fragment of a female steroid sex hormone receptor gene, and
determining whether there is a polymorphism in said nucleotide sequence wherein the presence of the polymorphism in said nucleotide sequence indicates that said individual has an increased predisposition to migraine compared to an individual without the polymorphism.

2. The method of claim 1, wherein said nucleotide sequence is of at least a fragment of exon 8 of a human estrogen receptor (ESR1) gene that encodes codon 594 of an estrogen receptor protein.

3. The method of claim 2, wherein the polymorphism is a guanine to adenine change at nucleotide 2014 of the ESR1 gene.

4. The method of claim 1, wherein said nucleotide sequence is of at least a fragment of a progesterone receptor gene, wherein said nucleotide sequence comprises a 306 base pair insertion in intron 7 of said progesterone receptor gene.

5. The method of claim 3, wherein the polymorphism is detected as a restriction fragment length polymorphism.

6. The method of claim 4, wherein said 306 base pair insertion is detected according to size.

7. The method of claim 1, wherein said sample comprises at least two nucleic acids from said individual, a first nucleic acid comprising a nucleotide sequence of at least a fragment of exon 8 of a human ESR1 gene and a second nucleic acid comprising a nucleotide sequence of at least a fragment of intron 7 of a human progesterone receptor gene.

8. The method of claim 7, wherein the first nucleic acid comprises a polymorphism that is a guanine to adenine change at nucleotide 2014 of the human ESR1 gene and/or the second nucleic acid comprises a 306 base pair insertion in intron 7 of the human progesterone receptor gene.

9. A method of determining whether an individual has a predisposition to migraine comprising
(a) obtaining a biological sample from said individual, said sample comprising
(i) a first nucleic acid that comprises a first nucleotide sequence of at least a fragment of a first female steroid sex hormone receptor gene; and
(ii) a second nucleic acid that comprises a second nucleotide sequence of at least a fragment of a second female steroid sex hormone receptor gene; and
(b) determining whether there is a polymorphism in each of said first and second nucleotide sequences,
wherein the presence of a polymorphism in said first nucleotide sequence of (i) and in said second nucleotide sequence of (ii) indicates that said individual has an increased predisposition to migraine compared to that of an individual having a polymorphism in (i) or (ii) alone.

10. The method of claim 9, wherein said first nucleotide sequence in (i) is of at least a fragment of exon 8 of a human estrogen receptor (ESR1) gene that encodes codon 594 of an estrogen receptor protein.

11. The method of claim 10, wherein the polymorphism is a guanine to adenine change at nucleotide 2014 of the ESR1 gene.

12. The method of claim 9, wherein said second nucleotide sequence in (ii) is of at least a fragment of a progesterone receptor gene, wherein said nucleotide sequence comprises a 306 base pair insertion in intron 7 of said progesterone receptor gene.

13. The method of any preceding claim, wherein migraine is migraine with aura or migraine without aura.

14. A kit for identifying a predisposition to migraine, said kit comprising one or more primers for nucleic acid sequence amplification of at least a fragment of a female sex steroid hormone receptor gene, and instructions to determine whether there is a polymorphism in said fragment.

15. The kit of claim 14, which comprises primers for nucleic acid sequence amplification of at least a fragment of exon 8 of a human ESR1 gene that encodes codon 594 of an estrogen receptor protein.

16. The kit of claim 15, wherein the kit further comprises a Btgl restriction endonuclease.

17. The kit of claim 14, which comprises primers for nucleic acid sequence amplification of at least a fragment of intron 7 of a human progesterone receptor gene.

18. A kit for identifying a predisposition to migraine, said kit comprising one or more primers for nucleic acid sequence amplification of:
(i) a first nucleic acid that comprises a nucleotide sequence of at least a fragment of a first female steroid sex hormone receptor gene; and
(ii) a second nucleic acid that comprises a nucleotide sequence of at least a fragment of a second female steroid sex hormone receptor gene.

19. The kit of claim 18, which comprises:
(a) primers for nucleic acid sequence amplification of at least a fragment of exon 8 of a human ESR1 gene that encodes codon 594 of an estrogen receptor protein; and
(b) primers for nucleic acid sequence amplification of at least a fragment of intron 7 of a human progesterone receptor gene.

20. The kit of claim 19, wherein the kit further comprises a Btgl restriction endonuclease.

21. A method of determining whether an individual has a predisposition to migraine comprising
isolating a progesterone receptor protein, or fragment thereof, and
determining whether said individual has a human progesterone receptor protein polymorphism,
wherein the presence of said polymorphism indicates an increased predisposition to migraine compared to an individual without the polymorphism.

22. The method of claim 21, wherein the progesterone receptor protein is detected according to an altered expression level that indicates said individual has a 306 base pair insertion in the human progesterone receptor gene.

23. The method of claim 1, wherein the determining step comprises amplification of said nucleic acid.

24. The method of claim 1, wherein the determining step comprises digesting said nucleic acid.

25. The method of claim 1, wherein the determining step comprises gel electrophoresis of said nucleic acid.


SEQUENCE LISTING (2 sequences)

SEQ ID NO: 1 - DNA, 18 bp, Arificial Sequence
  Oligonucleotide primer
gagacggaccaaagccac

SEQ ID NO: 2 - DNA, 22 bp, Artificial Sequence
  Oligonucleotide Primer
gccattggtgttggatgcatgc